SEQUENCE LISTING (18 sequences)

SEQ ID NO: 1 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 2 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 3 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 4 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 5 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 6 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 7 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 8 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 9 - protein, 5 aa, synthetic construct
GGGGS

SEQ ID NO: 10 - protein, 15 aa, synthetic construct
GGGGSGGGGSGGGGS

SEQ ID NO: 11 - protein, 19 aa, synthetic construct
MGWSCIILFLVATATGVHS

SEQ ID NO: 12 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, Homo sapiens [sequence omitted]

SEQ ID NO: 14 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 15 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 16 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 17 - protein, 158 aa, Mus musculus
SIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 18 - protein, 156 aa, Rattus sp.
SIQGTSLLTESCALSTYNDQSVSFVLENGCYVINVEDCGKNQEKDKVLLRYYESSFPAQSGDGVDGKKLMVNMSPIKDTDIWLNANDKDYSVELQKGDVSPPDQAFFVLHKKSSDFVSFECKNLPGTYIGVKDNQLALVEENDESCNNIMFKLSKM

(19) United States
(12) Patent Application Publication
(10) Pub. No.: US 2025/0066443 A1
(43) Pub. Date: Feb. 27, 2025

(54) COMPOSITIONS COMPRISING TRUNCATED INTERLEUKIN-33 AND INTERLEUKIN-2

(71) Applicant: Slate Bio, Inc., Charlottesville, VA (US)

(72) Inventors: Rahul SHARMA, Charlottesville, VA (US); Michelle SOLTERO HIGGIN, Holly Springs, NC (US); Thomas Charles BOONE, Newbury Park, CA (US); Randall Edward BURTON, Billerica, MA (US)

(21) Appl. No.: 18/943,283

(22) Filed: Nov. 11, 2024

Related U.S. Application Data
(63) Continuation of application No. PCT/US2023/066901, filed on May 11, 2023.
(60) Provisional application No. 63/340,777, filed on May 11, 2022.

(51) Int. Cl. C07K 14/55; A61K 38/20; C07K 14/54

(52) U.S. Cl. CPC C07K 14/55; A61K 38/20; A61K 38/2013; C07K 14/54; C07K 2319/02

(57) ABSTRACT

Disclosed herein are compositions comprising an interleukin-2 (IL-2) having at least 90% identity to SEQ ID NO: 13 and a truncated interleukin-33 (IL-33) comprising a sequence with at least 90% identity to SEQ ID NO: 1. The IL-2 and the truncated IL-33 can be domains of fusion proteins. The compositions or the fusion proteins can be used in methods for treating a disease or disorder, comprising administering to a subject in need thereof an effective amount of a pharmaceutical composition comprising the composition or the fusion protein. The disease or disorder can be an autoimmune disease or disorder, a disease or disorder characterized by inflammation, or a cancer.

### 1. RELATED APPLICATIONS

This application is a U.S. Continuation Application of PCT/US23/66901 filed on May 11, 2023, which claims priority to U.S. Provisional Application No. 63/340,777 filed on May 11, 2022; the contents of each of which are incorporated herein in their entirety.

### 2. SEQUENCE LISTING

The instant application contains a Sequence Listing which has been submitted electronically in XML format and is hereby incorporated by reference in its entirety. Said XML copy, created on May 9, 2023, is named 771185_000009_SL.xml and is 18,768 bytes in size.

### 3. BACKGROUND

Interleukin-2 (IL-2) is a cytokine which plays a role in maintenance of immune tolerance; regulation of T-helper (Th) cell function, especially Th2 function; the differentiation, survival, and function of Foxp3⁺CD4⁺ regulatory T (Treg) cells; and interacts with innate lymphoid cells (ILCs).

Interleukin-33 (IL-33) is a cytokine, which in its wild type form contains a nuclear localization signal at its N-terminus. Protease cleavage yields an 18 kDa C-terminal fragment, which may be referred to herein as truncated IL-33. Truncated IL-33 can stimulate innate lymphoid cells (ILCs), Th2 cells, Treg cells, macrophages, and dendritic cells, and thereby upregulate the expression of IL-2, IL-4, IL-5, and IL-13, and reduce pro-inflammatory Th1 and Th17 responses.

Fusion proteins comprising IL-2 domains and truncated IL-33 domains have been described in the art. See, for example, WO2018/140504, US2019/0365714, U.S. Pat. Nos. 9,840,545; 10,851,145; and US 20210261640; the disclosures of which are hereby incorporated by reference. Such fusion proteins can treat diseases such as autoimmune diseases and disorders, inflammation, etc. However, a number of such fusion proteins have been observed to have one or more undesirable properties, such as aggregation, fast clearance in vivo, or loss of IL-33 activity over time.

Accordingly, there is a need for fusion proteins comprising IL-2 domains and truncated IL-33 domains without one or more undesirable properties. IL-33 loses its activity due to spontaneous oxidation of certain residues. There is also a need for such fusion proteins to be easily expressed by recombinant organisms, easily purified, and have both IL-2 and IL-33activity comparable to wild type IL-2 and wild-type IL-33, respectively.

### 4. SUMMARY

The present disclosure provides compositions comprising an interleukin-2 (IL-2) having at least 90% identity to SEQ ID NO: 13 and a truncated interleukin-33 (IL-33) comprising a sequence with at least 90% identity to SEQ ID NO: 1.

The present disclosure also provides a fusion protein, comprising an interleukin-2 (IL-2) domain having at least 90% identity to SEQ ID NO: 13 and a truncated interleukin-33 (IL-33) domain comprising a sequence with at least 90% identity to SEQ ID NO: 1.

In the truncated IL-33 polypeptide or domain, at least one of N60, C97, C116, C121, and C148 of SEQ ID NO: 1 can be substituted by a single amino acid. For example, N60 of SEQ ID NO: 1 can be substituted by Ser or Asp, C97 of SEQ ID NO: 1 can be substituted by Gly, and/or C116 of SEQ ID NO: 1 can substituted by Phe. The truncated IL-33 polypeptide or domain can have a sequence selected from the group consisting of SEQ ID NO: 3, SEQ ID NO: 4, SEQ ID NO: 5, SEQ ID NO: 6, SEQ ID NO: 7, and SEQ ID NO: 8; or selected from the group consisting of SEQ ID NO: 3, SEQ ID NO: 4, or SEQ ID NO: 7.

The fusion protein can further comprise an optional linker, such as an oligopeptide linker. For example, a linker can comprise GGGGS (SEQ ID NO: 9). The linker can have the sequence GGGGSGGGGSGGGGS (SEQ ID NO: 10).

The IL-2 polypeptide or domain can have a sequence of SEQ ID NO: 13, SEQ ID NO: 14; SEQ ID NO: 15; or SEQ ID NO: 16.

The fusion protein can further comprise an optional signal peptide. The signal peptide can have the sequence of SEQ ID NO: 11.

In the fusion protein, the domains can be, in order from N-terminus to C-terminus, an optional signal peptide; the interleukin-2 (IL-2) domain; an optional linker; and the truncated interleukin-33 (IL-33) domain.

The present disclosure also includes a pharmaceutical composition, comprising a composition or a fusion protein as described herein.

The pharmaceutical composition can further comprise a pharmaceutically-acceptable carrier, and optionally an additional therapeutic agent.

The disclosure includes a polynucleotide comprising a nucleic acid sequence encoding at least a portion of a fusion protein described herein.

In another aspect, the disclosure includes a vector comprising the polynucleotide operatively coupled to a promoter.

In yet another aspect, the disclosure includes a recombinant host cell comprising the polynucleotide or the vector.

The disclosure includes a method for treating a disease or disorder, comprising administering to a subject in need thereof an effective amount of a pharmaceutical composition described herein.

The disease or disorder can be acute kidney injury, ankylosing spondylitis, autoimmune cardiomyopathy, autoimmune hemolytic anemia, autoimmune hepatitis, autoimmune lymphoproliferative syndrome, autoimmune pancreatitis, autoimmune polyendocrine syndrome, autoimmune urticaria, autoimmune uveitis, Crohn's disease, dermatomyositis, diabetic nephropathy, diabetic retinopathy, graft versus host (GVH) disease, Hashimoto's thyroiditis, idiopathic inflammatory bowel disease (IBD), inflammatory demyelinating diseases, Inflammatory neuropathies, insulitis, interstitial cystitis, juvenile idiopathic arthritis, lupus, lupus erythematosus, lupus glomerulonephritis, lupus nephritis, cutaneous lupus, IgA nephropathy, membranous nephropathy (MPGN), microscopic colitis, multiple sclerosis, myasthenia gravis, obesity, pancreatitis, polymyositis, primary biliary cirrhosis (PBC), primary sclerosing cholangitis, progressive inflammatory neuropathy, renal ischemia reperfusion injury, rheumatoid arthritis, Sjogren's syndrome, systemic lupus erythematosus, transplant rejection, type 1 diabetes, type 2 diabetes, ulcerative colitis, vasculitis, psoriasis, atopic dermatitis, pemphigus vulgaris, or Wegener's granulomatosis.

The disease or disorder can be a cancer. The cancer can be acute lymphoblastic leukemia (ALL), acute myeloid leukemia (AML), acute myelogenous leukemia, adrenocortical carcinoma, anal cancer, appendix cancer, astrocytoma, basal cell carcinoma, B-cell lymphoma, brain cancer, bile duct cancer, bladder cancer, bone cancer, breast cancer, bronchial tumor, carcinoma of unknown primary origin, cardiac tumor, cervical cancer, chordoma, chronic lymphocytic leukemia, chronic myelogenous leukemia, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphocytic leukemia, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

The cancer can be melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer.

Depending upon the IL-2 domain, the method can stimulate proliferation and/or activation of T-regulatory (Treg) cells, T-helper2 (Th2) cells, innate lymphoid cells (ILC), cytotoxic T cells, natural killer (NK) cells, NK-T cells, ST2+cells, dendritic cells, and/or macrophages.

Depending upon the IL-2 domain, the method can either promote anti-inflammatory M2 macrophages and inhibit pro-inflammatory M1 macrophages, or activate M1macrophages to induce inflammation. The M2 macrophages can inhibit inflammation in a tissue or organ selected from the group consisting of pancreas, liver, kidneys, adipose tissue, salivary glands, central nervous system (CNS), and related organs. The M1 macrophages can promote anti-tumor immune response either systemically or locally.

Depending on the IL-2 domain, the method can either upregulate or down-regulate the expression of co-stimulatory molecules (including and not limited to CD80 (B7-1), CD86 (B7-2), CD40, ICOS, MHC-I, MHC-II, PD1, PD-L1, GITR, BAFF-R, Ox40, 41BB, DR3, CR2) on the antigen-presenting cells including and not limited to dendritic cells, macrophages, B-cells, innate lymphoid cells, NK cells, epithelial cells, endothelial cells or stromal cells.

The method can further comprise administering an effective amount of a second therapy for the disease or disorder to the subject, wherein the second therapy is not a fusion protein described herein.

The second therapy can be a cell therapy or a gene therapy.

In another aspect, the disclosure includes a method for stimulating proliferation and/or activation of T-regulatory (Treg) cells of a subject, comprising: isolating T cells from the subject; and contacting the isolated T cells with an effective amount of a composition, fusion protein, recombinant host cell, or cell expressing at least one of IL-2 or truncated IL-33, as described herein.

The disclosure also includes a kit, comprising a pharmaceutical composition described herein; and instructions for performing a method for treating a disease or disorder characterized by inflammation, the method comprising administering to a subject in need thereof an effective amount of the pharmaceutical composition.

### 5. BRIEF DESCRIPTION OF THE DRAWINGS

FIG. 1 shows sequences of wild type human IL-2 (SEQ ID NO: 13) and truncated wild type human IL-33 (SEQ ID NO: 1). In the IL-2 sequence, two residues each of interest in some embodiments, N88 and C125, are emphasized. In the truncated IL-33 sequence, three residues each of interest in some embodiments, N60, C97, and C116, are emphasized.

FIG. 2 shows a sequence alignment between wild type human truncated IL-33 (SEQ ID NO: 1), mouse truncated IL-33 (SEQ ID NO: 17), and rat truncated IL-33 (SEQ ID NO: 18). In the human sequence, an N-glycosylation site comprising N60, and the four wild type Cys residues, C97, C116, C121, and C148, are highlighted. In the mouse and rat sequences, Cys residues aligned with human Cys residues are emphasized.

FIG. 3 shows a graph of IL-2 activity of a fusion protein comprising an IL-2 domain and a truncated IL-33 domain with a N60D point substitution, as described in Example 1.

FIG. 4 shows a graph of IL-2 activity of a fusion protein comprising an IL-2 domain and a truncated IL-33 domain with a C97G point substitution, as described in Example 2.

FIG. 5 shows a graph of IL-33 activity of a fusion protein comprising an IL-2 domain and a truncated IL-33 domain with a C97G point substitution, as described in Example 2.

FIG. 6 shows expression in a recombinant host cell of a fusion protein comprising an IL-2 domain and a truncated IL-33 domain with a C116F point substitution, as described in Example 3.

FIG. 7 shows purification of a fusion protein comprising an IL-2 domain and a truncated IL-33 domain with a C116F point substitution, as described in Example 3.

FIG. 8 shows a graph of IL-2 activity of a fusion protein comprising an IL-2 domain and a truncated IL-33 domain with a C116F point substitution, as described in Example 3.

FIG. 9 shows a graph of IL-33 activity of a fusion protein comprising an IL-2 domain and a truncated IL-33 domain with a C116F point substitution, as described in Example 3.

FIG. 10 shows a graph of IL-33 activity of a fusion protein comprising an IL-2 domain and a truncated IL-33 domain with a C116F point substitution after 0, 3, or 7 days of storage at 4° C., 25° C., or 37° C., as described in Example 3.

FIG. 11A is a graph showing the IL2 activity of wild type IL2, wild type fusion protein IL233 74291-1 (SLT1), and engineered IL233 fusion proteins 7429-2 (SLT2), 7429-3 (SLT3), 7429-4 (SLT4), 7429-5 (SLT5), and 7429-6 (SLT6). FIG. 11B is a graph showing the IL33 activity of wild type IL33, wild type fusion protein IL233 74291-1 (SLT1), and engineered IL233 fusion proteins 7429-2 (SLT2), 7429-3 (SLT3), 7429-4 (SLT4), 7429-5 (SLT5), and 7429-6 (SLT6).

FIG. 12A is a graph showing the IL2 activity of wild type IL2, wild type fusion protein IL233 74291-1 (SLT1), and engineered IL233 fusion proteins 7429-2 (SLT2), 7429-3 (SLT3), and 7429-4 (SLT4). FIG. 12B is a graph showing the IL33 activity of wild type IL33, wild type fusion protein IL233 74291-1 (SLT1), and engineered IL233 fusion proteins 7429-2 (SLT2), 7429-3 (SLT3), and 7429-4 (SLT4).

FIG. 13A is a graph showing the IL2 activity of wild type IL2, wild type fusion protein IL233 74291-1 (SLT1), and engineered IL233 fusion proteins 7429-5 (SLT5) and 7429-6 (SLT6). FIG. 13B is a graph showing the IL33 activity of wild type IL33, wild type fusion protein IL233 74291-1 (SLT1), and engineered IL233 fusion proteins 7429-5 (SLT5) and 7429-6 (SLT6).

FIG. 14A is a graph showing the IL2 activity of wild type IL2, and engineered IL233 fusion proteins 7429-2 (SLT2) and 7429-4 (SLT4). FIG. 14B is a graph showing the IL33 activity of wild type IL33, and engineered IL233 fusion proteins 7429-2 (SLT2) and 7429-4 (SLT4).

FIG. 15 is a graph showing IL233 fusion protein (SLT-518(h4)) promotes dose dependent ST2+ Treg proliferation in fresh human PBMCs. IL-2 and SLT518(h4) was added at 25 ng/ml and 50 ng/ml and the percentage of ST2 Tregs were measured.

FIG. 16A is a schematic that illustrates study in a mouse model wherein mice received daily equimolar injections of SLT-518(h4) over five days. FIG. 16B is a graph of blood ST2+ Tregs as measured at day 6. FIG. 16C is a graph of tissue ST2+ Tregs as measured in lymph nodes, kidneys, and lungs at day 8, and cytokines. The results show that human SLT-518(h4) (IL233 fusion protein) signals in vivo and drives Tregs into tissues.

FIG. 17 is a composite showing schematically that SLT-518(h4), an IL233 fusion protein, drives the local tissue environment to a more tolerogenic phenotype in mice. Supporting data shows that natural killer (NK) cells and co-stimulatory molecules on dendritic cells (DC) decrease after administration of the IL233 fusion protein. M2 macrophages, ST2+ Tregs, and ratios of IL10/TNFa, Treg/Th17, ILC2 all increase after administration of the IL233 fusion protein.

FIG. 18 is a graph showing treatment with SLT-518(h4) (referred to as SLT4) promoted dramatic expansion of ST2+ Treg cells in healthy non-human primates.

FIG. 19 is a graph showing selective expansion of Total and ST2+ Tregs without concomitant expansion of other proinflammatory T-cells, cytotoxic T-cells, B-cells or natural killer cells in healthy non-human primates (NHP) upon administration of SLT-518.

FIG. 20A is a graph showing the percentage of Tregs in non-human primates over time (21 days) after administration of five daily doses of 100 ug/kg of either wild type fusion protein IL233 74291-1 (SLT1), engineered IL233 fusion proteins 7429-2 (SLT2) or 7429-4 (SLT4). FIG. 20B is a graph showing the fold change over time (21 days) after administration of five daily doses of 100 ug/kg of either wild type fusion protein IL233 74291-1 (SLT1), engineered IL233 fusion proteins 7429-2 (SLT2) or 7429-4 (SLT4).

FIG. 21A and FIG. 21B are graphs showing SLT-518(h4) induced superior expansion of ST2+ Tregs, thereby providing a wider therapeutic margin relative to clinical doses of IL-2.

FIG. 22 is a graph showing SLT-518(h4) exhibited a dramatic expression of ST2+ Tregs in the blood and remained elevated in the tissue for over 28 days, well beyond the levels observed in the blood.

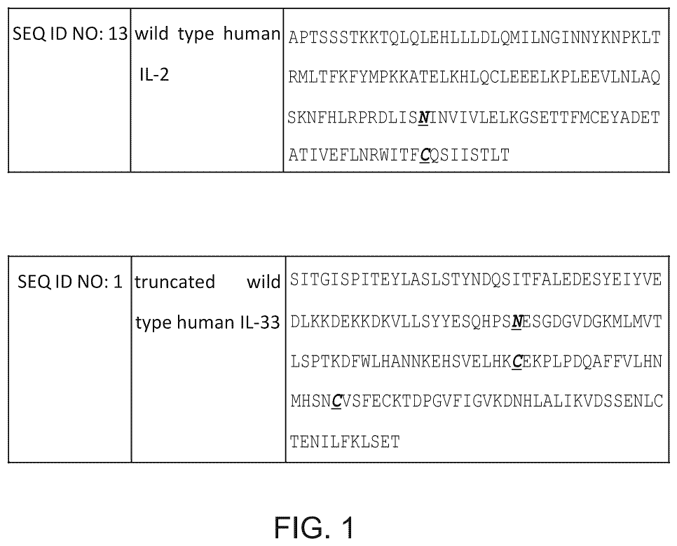

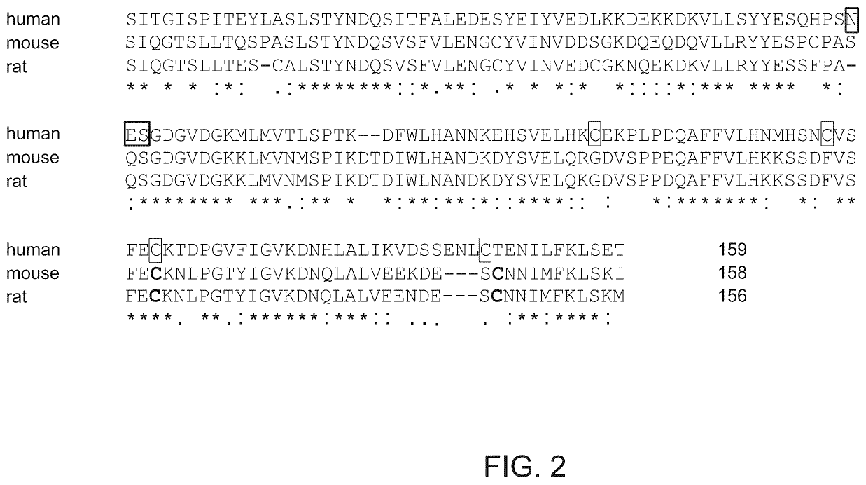

### 6. DETAILED DESCRIPTION

This disclosure relates to compositions or fusion proteins comprising an IL-2 polypeptide or domain, and a truncated IL-33 polypeptide or domain. The fusion of IL-2 and IL-33 is key to enabling increased potency, tissue targeting and avidity. ST2 is the receptor for IL-33 and is expressed on the surface of a subset of regulatory T cells (Treg). This subset is more durable and active in modulating inflammatory suppression in tissues at the site of disease. Because IL-33 is naturally released in tissues, its function not only helps to expand ST2+ Treg cells, but other ST2+ cells and promotes a local tolerogenic tissue micro-environment. Treatment with the fusion proteins and compositions described herein thus results in durable disease resolution.

In the fusion proteins, the domains can be joined by a linker, such as an oligopeptide linker. The compositions or fusion proteins can have one or more advantageous properties, including but not limited to any one or more of reduced aggregation, improved stability during storage, retention of IL-33 activity after storage, easier production by recombinant organisms, easier purification, IL-2 activity comparable to wild type IL-2, IL-33 activity comparable to wild-type IL-33, and prolongation of IL-33 activity post administration. Though not wishing to be bound by theory, such advantageous properties can arise from site-specific mutations in IL-2 domains and/or truncated IL-33 domains that reduce aggregation, such as by replacing cysteine residues with other residues that are not capable of forming disulfide linkages.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

Additional description of the methods and guidance for the practice of the methods are provided herein.

### A. EMBODIMENTS

### i. Compositions

In some embodiments, the present disclosure relates to a composition. The composition can comprise an interleukin-2 (IL-2) and a truncated interleukin-33 (IL-33). In the composition, the interleukin-2 (IL-2) can comprise SEQ ID NO: 13, or have at least 90%, 91%, 92%, 93%, 94%, 95%, 96%, 97%, 98%, or 99% sequence identity to SEQ ID NO: 13. Alternatively or in addition, the truncated interleukin-33 (IL-33) can comprise SEQ ID NO: 1, or have at least 90%, 91%, 92%, 93%, 94%, 95%, 96%, 97%, 98% or 99% sequence identity to SEQ ID NO: 1.

The IL-2 and the truncated IL-33 will generally be described herein in the context of fusion proteins. However, the person of ordinary skill in the art will understand that the descriptions of IL-2 and truncated IL-33 domains will apply equally to IL-2 and truncated IL-33 polypeptides in the compositions, except for description specific to fusion proteins (e.g., arrangement of domains in a fusion protein or the like).

### ii. Fusion Proteins

In some embodiments, the present disclosure relates to a fusion protein. The fusion protein can comprise an interleukin-2 (IL-2) domain and a truncated interleukin-33 (IL-33) domain. The fusion protein can comprise an interleukin-2 (IL-2) domain comprising SEQ ID NO: 13, or an IL-2 domain having at least 90%, 91%, 92%, 93%, 94%, 95%, 96%, 97%, 98%, or 99% sequence identity to SEQ ID NO: 13; and a truncated interleukin-33 (IL-33) domain comprising SEQ ID NO:1, or a truncated IL-33 domain having at least 90%, 91%, 92%, 93%, 94%, 95%, 96%, 97%, 98%, or 99% sequence identity to SEQ ID NO: 1.

In some embodiments, the fusion protein can comprise an optional linker between a first and a second domain.

In some embodiments, the fusion protein can comprise more than one IL-2 domain, more than one truncated IL-33 domain, or both.

### IL-2 Domains

The IL-2 domain of the fusion protein can be identical to or a variant of the wild type IL-2 sequence of any organism. In one aspect, the IL-2 domain is at least 90% identical to the wild type IL-2 sequence of the organism to which the fusion protein is intended for administration in the methods described herein.

In one embodiment, the IL-2 domain comprises a sequence having at least 90% identity to SEQ ID NO: 13, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 13. The IL-2 domain of the fusion protein can differ from SEQ ID NO: 13 by zero, one, two, three, four, five, six, seven, eight, nine, ten, eleven, twelve, or thirteen positions.

The sequence of the IL-2 domain can comprise a number of variants of SEQ ID NO: 13. For example, the IL-2 domain can comprise a point mutation of the Asn at position 88 (N88) of SEQ ID NO: 13. IL-2 domains comprising a point mutation of N88 can exhibit one or more advantageous properties, including, but not limited to, higher stability during storage and/or higher activity than a corresponding IL-2 domain lacking the point mutation of N88.

For a particular example, the IL-2 domain can comprise a point substitution from Asn to Asp at position 110 (N88D) of SEQ ID NO: 13. A particular IL-2 domain containing the N88D substitution has the sequence of SEQ ID NO: 14.

In another embodiment, the IL-2 domain can comprise a point mutation of the Cys at position 125 (C125) of SEQ ID NO: 13. IL-2 domains comprising a point mutation of C125 are less likely to form disulfide linkages within the IL-2 domain and/or to Cys residues in the IL-33 domain, and can exhibit one or more advantageous properties, including but not limited to, reduced aggregation, slower clearance in vivo, or higher IL-2 activity than a corresponding IL-2 domain lacking the point mutation of C125.

In a specific embodiment, the IL-2 domain can comprise a point substitution from Cys to Ser at position 125 (C125S) of SEQ ID NO: 13. A particular IL-2 domain containing the C125S substitution has the sequence of SEQ ID NO: 15.

Different IL-2 mutations can decrease the interaction of IL-2 with IL2Rα (CD25) and/or increase the interaction of IL-2 with IL2Rβ (CD122), allowing differential activation of NK and CD8 T cells compared to Tregs.

Any two or more of the deletions or substitutions within the IL-2 domain as described above can be combined. In one embodiment, the IL-2 domain can comprise point mutations of both the Asn at position 88 and the Cys at position 125 of SEQ ID NO: 13. For a particular example, the IL-2 domain can comprise both point substitutions N88D and C125S of SEQ ID NO: 13. A particular IL-2 domain containing both the N88D and the C125S substitution has the sequence of SEQ ID NO: 16.

### Linkers

The optional linker can be any moiety capable of forming at least two covalent bonds to the N-terminus of a polypeptide, the C-terminus of a polypeptide, or an amino acid side chain of a polypeptide. Preferably, the linker is an oligopeptide sequence, comprising one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, or 30 amino acids. An oligopeptide can form a covalent bond to the C-terminus of a polypeptide and the N-terminus of (typically another) a polypeptide.

Preferably, the amino acids are encoded by codons of the canonical genetic code. This can allow a fusion protein as a whole to be encoded by a single polynucleotide and be transcribed and translated as a unit, thereby simplifying manufacture of the fusion protein.

When the linker is an oligopeptide, the amino acids thereof can be chosen to allow the linker to be flexible, generally unlikely to form secondary structures, generally unlikely to interfere with the tertiary structures of the domains linked by the linker, generally unlikely to link with any domain of the fusion protein other than at the C-terminus and N-terminus described above, and generally non-immunogenic. An oligopeptide linker comprising at least about 75% Gly residues can be used.

Particular amino acid residues of an oligopeptide linker can be chosen to adjust the isoelectric point (pI) of the fusion protein, which can be useful in increasing the yield, purity, storage, or efficacy of the fusion protein in manufacture or use.

For example, the linker can comprise GGGGS (SEQ ID NO: 9). In another aspect, the linker can comprise the sequence GGGGSGGGGSGGGGS (SEQ ID NO: 10).

In some embodiments, the fusion protein can comprise an optional additional linker, whereby the fusion protein can dimerize or multimerize. The additional linker can be an oligopeptide linker, such as those described herein.

### IL-33 Domains

The IL-33 domain can have a wild type, variant, truncated, or truncated variant sequence of IL-33 from any species. In one aspect, the IL-33 domain comprises a sequence that is at least 90%, 91%, 92%, 93%, 94%, 95%, 96%, 97%, 98%, or 99% identical to the wild type IL-33 sequence of the organism to which the fusion protein is intended for administration in the methods described herein.

Wild type human truncated IL-33 has the sequence of SEQ ID NO: 1. Other organisms have truncated IL-33 comprising residues homologous to the human truncated IL-33 of SEQ ID NO: 1.

In one aspect, the truncated IL-33 domain comprises a sequence having at least 90% identity to SEQ ID NO: 1, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 1. The truncated IL-33 domain of the fusion protein can differ from SEQ ID NO: 1 at zero, one, two, three, four, five, six, seven, eight, nine, ten, eleven, twelve, thirteen, fourteen, or fifteen positions.

In some embodiments, the truncated IL-33 domain can comprise a sequence with at least 90%,/91%, 92%, 93%, 94%, 95%, 96%, 97%, 98%, or 99% identity to SEQ ID NO: 1, wherein one or more possible sites of glycosylation or disulfide linkage (e.g., N60, C97, C116, C121, and C148) are deleted or substituted by one or more amino acids.

A number of variants of SEQ ID NO: 1 can be particularly considered for the sequence of the truncated IL-33 domain.

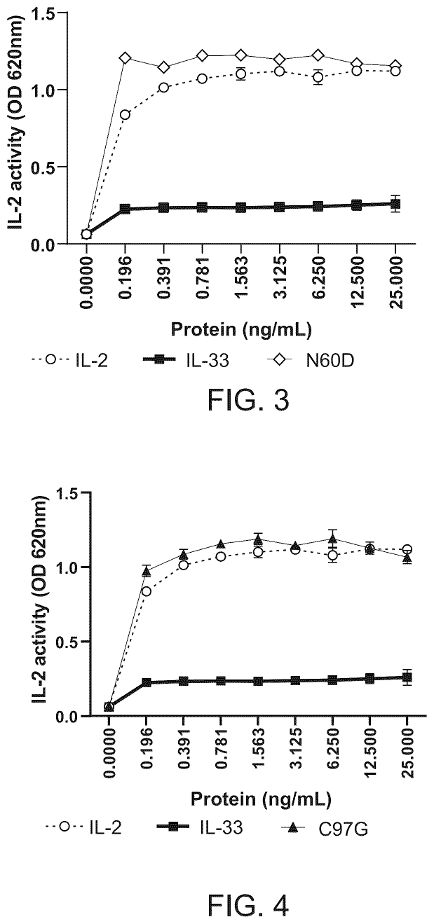

In one aspect, the truncated IL-33 domain can comprise a point mutation of the Asn at position 60 (N60) of SEQ ID NO: 1. As shown in FIG. 3, N60 is present in human truncated IL-33, but is not present in the homologous position in mouse or rat truncated IL-33. Truncated IL-33 domains comprising a point mutation of N60 are less susceptible to glycosylation, and can exhibit one or more advantageous properties including, but not limited to, reduced aggregation, slower clearance in vivo, or higher activity than a corresponding truncated IL-33 domain lacking the point mutation of N60. In a particular example, the truncated IL-33 domain can comprise a point substitution at position 60 from Asn to Ser (N60S), or from Asn to Asp (N60D). A particular truncated IL-33 domain comprising the N60S point substitution can have the sequence of SEQ ID NO: 2.

In one embodiment the truncated IL-33 domain can comprise a point mutation of the Cys at position 116 (C116) of SEQ ID NO: 2. Truncated IL-33 domains comprising a point mutation of C116 are less likely to form disulfide linkages within the truncated IL-33 domain and/or to Cys residues in the IL-2 domain, and can exhibit one or more advantageous properties including, but not limited to, reduced aggregation, slower clearance in vivo, or higher activity than a corresponding truncated IL-33 domain lacking the point mutation of C116.

In one embodiment, the truncated IL-33 domain can comprise a point substitution from Cys to Phe at position 116 (C116F). The C116F point substitution was based on the observation that mouse and rat truncated IL-33 comprise a Phe at the position homologous to human wild type C116, as shown in FIG. 3.

The C116F point substitution provides improvement to expression and purification of fusion proteins comprising both IL-2 domains and truncated IL-33 domains, and the activities of the truncated IL-33 domains and the IL-2 domains are essentially the same as wild type IL-33 alone and IL-2 alone, respectively.

A particular truncated IL-33 domain comprising the C116F point substitution can have the sequence of SEQ ID NO: 3.

In one aspect, the truncated IL-33 domain can comprise a point mutation of the Cys at position 97 (C97) of SEQ ID NO: 2. Truncated IL-33 domains comprising a point mutation of C97 are less likely to form disulfide linkages and can exhibit one or more of the desirable properties discussed with reference to C116.

In another aspect, the truncated IL-33 domain can comprise a point substitution from Cys to Gly at position 97 (C97G). The C97G point substitution was based on the observation that mouse and rat truncated IL-33 comprise a Gly at the position homologous to human wild type C97, as shown in FIG. 3.

In the context of the C97G point substitution, the activities of both the truncated IL-33 domains and the IL-2 domains are essentially the same as wild type IL-33 alone and IL-2 alone, respectively.

A particular truncated IL-33 domain comprising the C97G point substitution can have the sequence of SEQ ID NO: 4.

Any two or more of the deletions or substitutions within the truncated IL-33domain as described above can be combined. For example, the truncated IL-33 domain can comprise both N60S and C116F (SEQ ID NO: 5); both N60S and C97G (SEQ ID NO: 6); both C116F and C97G (SEQ ID NO: 7); or all three of N60S, C116F, and C97G (SEQ ID NO: 8).

Accordingly, in some embodiments, the truncated IL-33 domain has a sequence selected from the group consisting of SEQ ID NO: 3, SEQ ID NO: 4, SEQ ID NO: 5, SEQ ID NO: 6, SEQ ID NO: 7, and SEQ ID NO: 8.

In some embodiments, the truncated IL-33 domain has a sequence from the group consisting of SEQ ID NO: 3, SEQ ID NO: 4, or SEQ ID NO: 7.

### Signal Peptides

The fusion protein can also comprise a signal peptide, such as at the N-terminus of the fusion protein. The signal peptide can be chosen from signal peptides known for use with the N-terminal domain of the fusion protein, or can be selected based on rational design or routine experimentation.

In one embodiment, if the N-terminal domain of the fusion protein is the IL-2 domain, the signal peptide can have the sequence of SEQ ID NO: 11.

### Linkage of Domains

The various domains of a fusion protein can be linked through any desired reactive sites of the various domains, either directly or via the reactive moieties of the optional linker. In some embodiments, wherein the linker is an oligopeptide, the various domains can be linked through their N- and/or C-termini to the complementary terminus of the linker. A fusion protein can comprise a single continuous polypeptide sequence. Such a fusion protein can be prepared from a single polynucleotide coding region encoding the entire sequence of the fusion protein, by well-known transcriptional and translational processes.

A fusion protein comprising a single continuous polypeptide sequence can comprise the various domains in any desired order. For example, the fusion protein can comprise any of the IL-2 domain or the truncated IL-33 domain at its N-terminus; the linker; and the remaining domain at its C-terminus.

In one particular embodiment, the fusion protein can comprise, in order from N-terminus to C-terminus, a signal peptide; an interleukin-2 (IL-2) domain; a linker; and a truncated interleukin-33 (IL-33) domain.

In one particular embodiment, the fusion protein can comprise, in order from N-terminus to C-terminus, a signal peptide; a truncated IL-33 domain; a linker; and an IL-2 domain.

In one particular embodiment, the fusion protein can comprise, in order from N-terminus to C-terminus, a signal peptide; an IL-2 domain; and a truncated IL-33 domain.

In one particular embodiment, the fusion protein can comprise, in order from N-terminus to C-terminus, a signal peptide; a truncated IL-33 domain; and an IL-2 domain.

In one particular embodiment, the fusion protein can comprise, in order from N-terminus to C-terminus, an IL-2 domain; a linker; and a truncated IL-33 domain.

In one particular embodiment, the fusion protein can comprise, in order from N-terminus to C-terminus, a truncated IL-33 domain; a linker; and an IL-2 domain.

In one particular embodiment, the fusion protein can comprise, in order from N-terminus to C-terminus, a truncated IL-33 domain and an IL-2 domain.

In one particular embodiment, the fusion protein can comprise, in order from N-terminus to C-terminus, an IL-2 domain and a truncated IL-33 domain.

In another particular embodiment, the fusion protein can comprise two polypeptides linked by an additional linker between amino acid side chains. The first polypeptide can comprise, in order from N-terminus to C-terminus, an optional signal peptide and an IL-2 domain; the second polypeptide can comprise a truncated IL-33 domain; and the first and second polypeptides can be linked by an additional linker between side chains of an amino acid in the IL-2 domain of the first polypeptide and an amino acid in the truncated IL-33 domain of the second polypeptide.

### Manufacture of Fusion Proteins

The fusion proteins described herein can be manufactured by any known method. Generally, any polypeptide component of the fusion protein can be synthesized by chemical or biochemical methods, both of which are generally known to a person of ordinary skill in the art.

In biochemical methods of polypeptide synthesis, a polynucleotide comprising a nucleic acid sequence encoding the polypeptide of interest (e.g., the fusion protein or a portion thereof) is incorporated into a vector, wherein the polynucleotide is operatively coupled to a promoter. The vector can comprise other sequences, such as non-promoter control sequences, an origin of replication, sequences engineered for insertion of the polynucleotide into the vector, etc. The promoter can be chosen such that when the vector is introduced into a host cell by recombinant DNA techniques, the promoter is recognized by the host cell's endogenous RNA polymerase and, either constitutively or upon induction by one or more changes in process conditions, produces RNA transcripts. The RNA transcripts are then translated by the host cell's ribosomes to produce the polypeptide of interest (e.g., the fusion protein or the portion thereof).

Another route of polypeptide synthesis is the introduction of a polynucleotide expressing the polypeptide into cells of a subject in need thereof. The polynucleotide can comprise mRNA or DNA. The subject's cells can transcribe the DNA and/or translate the mRNA into the polypeptide of interest.

In embodiments wherein the fusion protein comprises multiple polypeptides, each polypeptide can be produced separately and the fusion protein assembled by peptide synthase reactions or by non-amino acid backbone linkages, such as by one or more non-oligopeptide linkers, by cross-polypeptide linkages through amino acid side chains of oligopeptide linkers, and/or by chemical conjugation.

In some embodiments, a composition comprising an IL-2 polypeptide and a truncated IL-33 polypeptide can be an intermediate stage in the preparation of a fusion protein comprising an IL-2 domain and a truncated IL-33 domain, and optionally a linker.

### iii. Pharmaceutical Compositions

In some embodiments, the present disclosure relates to a pharmaceutical composition. The pharmaceutical composition can comprise any composition or a fusion protein described above. In some embodiments, the pharmaceutical composition can further comprise a pharmaceutically-acceptable carrier. Alternatively or in addition, the pharmaceutical composition can further comprise an additional therapeutic agent.

### Pharmaceutically Acceptable Carriers

The pharmaceutical compositions can comprise a pharmaceutically-acceptable carrier. Pharmaceutically-acceptable carriers include physiologically tolerable or acceptable diluents, excipients, solvents, or adjuvants. The compositions are preferably sterile and nonpyrogenic.

### Additional Therapeutic Agents

In embodiments, additional therapeutic agents, by which is meant one or more therapeutic agents other than the fusion proteins described above, can be included in the pharmaceutical compositions. The additional therapeutic agents can include, but are not limited to, cytotoxic agents, anti-angiogenic agents, pro-apoptotic agents, antibiotics, hormones, hormone antagonists, chemokines, drugs, prodrugs, toxins, or enzymes.

Particular therapeutic agent(s) can be chosen in view of a disease or disorder intended to be treated by the pharmaceutical composition, among other parameters that will be known to a person of ordinary skill in the art.

The pharmaceutical compositions can also contain minor amounts of nontoxic auxiliary pharmaceutical substances, excipients, and/or additives, such as wetting agents, emulsifying agents, and/or pH buffering agents, among others known to a person of ordinary skill in the art.

If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions.

### iv. Treatment of Diseases and Disorders

In some embodiments, the present disclosure relates to the treatment of diseases and disorders by methods comprising administering to a subject in need thereof an effective amount of a pharmaceutical composition comprising a composition or a fusion protein described herein. The disease or disorder can be characterized by an inflammatory condition. In embodiments, the inflammatory condition can be present in a tissue or organ selected from the group consisting of pancreas, liver, kidneys, adipose tissue, salivary glands, central nervous system (CNS), and related organs.

Though not wishing to be bound by theory, the fusion protein can stimulate proliferation and/or activation of T-regulatory (Treg) cells, T-helper2 (Th2) cells, innate lymphoid cells (ILC), cytotoxic T cells, natural killer (NK) cells, NK-T cells, ST2+ cells, dendritic cells, and/or macrophages; promote anti-inflammatory M2 macrophages; and/or inhibit pro-inflammatory M1 macrophages.

Though not wishing to be bound by theory, the fusion protein, depending on the IL-2 domain, the method can either upregulate or down-regulate the expression of co-stimulatory molecules (including and not limited to CD80 (B7-1), CD86 (B7-2), CD40, ICOS, MHC-I, MHC-II, PD1, PD-L1, GITR, BAFF-R, Ox40, 41BB, DR3, CR2) on the antigen-presenting cells including and not limited to dendritic cells, macrophages, B-cells, innate lymphoid cells, NK cells, epithelial cells, endothelial cells or stromal cells.

### Diseases and Disorders

In some embodiments, the disease or disorder can be an autoimmune disorder. In some embodiments, the disease or disorder can be acute kidney injury, ankylosing spondylitis, autoimmune cardiomyopathy, autoimmune hemolytic anemia, autoimmune hepatitis, autoimmune lymphoproliferative syndrome, autoimmune pancreatitis, autoimmune polyendocrine syndrome, autoimmune urticaria, autoimmune uveitis, Crohn's disease, dermatomyositis, diabetic nephropathy, diabetic retinopathy, graft versus host (GVH) disease, Hashimoto's thyroiditis, idiopathic inflammatory bowel disease (IBD), and inflammatory demyelinating disease, inflammatory neuropathy, insulitis, interstitial cystitis, juvenile idiopathic arthritis, lupus, lupus erythematosus, lupus glomerulonephritis, lupus nephritis, IgA nephropathy, membranous nephropathy (MPGN), microscopic colitis, multiple sclerosis, myasthenia gravis, obesity, pancreatitis, polymyositis, primary biliary cirrhosis, primary sclerosing cholangitis, progressive inflammatory neuropathy, renal ischemia reperfusion injury, rheumatoid arthritis, Sjogren's syndrome, systemic lupus erythematosus, transplant rejection, type 1 diabetes, type 2 diabetes, ulcerative colitis, vasculitis, and Wegener's granulomatosis.

In some embodiments, the disease or disorder is a cancer. For example, the cancer can be any one of acute lymphoblastic leukemia (ALL), acute myeloid leukemia (AML), acute myelogenous leukemia, adrenocortical carcinoma, anal cancer, appendix cancer, astrocytoma, basal cell carcinoma, B-cell lymphoma, brain cancer, bile duct cancer, bladder cancer, bone cancer, breast cancer, bronchial tumor, carcinoma of unknown primary origin, cardiac tumor, cervical cancer, chordoma, chronic lymphocytic leukemia, chronic myelogenous leukemia, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphocytic leukemia, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

The cancer can be selected from the group consisting of melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, and lung cancer.

### Administration Techniques

The pharmaceutical compositions can be delivered to the subject via any route by which the fusion protein can arrive at a target site, e.g., a tissue or organ at which inflammation, cancerous cells, or other phenomena associated with the disease or disorder are present. The route can be local or systemic. For example, the pharmaceutical composition can be systematically delivered intravenously or intraarterially, among other routes; or locally delivered subcutaneously, intramuscularly, intraorganally, or intratumorally, among other routes. Another route of administration is the introduction of mRNA expressing the polypeptide into cells of the subject. The subject's cells can translate the mRNA into the polypeptide of interest, similarly to the use of mRNA in vaccines against SARS/CoV2.

A particular route of administration can be selected based on the target site, the disease or disorder, the effective amount of the fusion protein, other components of the pharmaceutical composition, and/or a second therapy (if any) included in the pharmaceutical composition, among other considerations. A particular route of administration can be selected as a routine matter by a person of ordinary skill in the art having the benefit of the present disclosure.

### Adjunct Therapies

For any disease or disorder, the method can further comprise administering adjunct therapy, wherein the adjunct therapy does not comprise the fusion protein.

For example, when the disease or disorder is cancer, the adjunct therapy can comprise surgical resection, radiation, chemotherapy, checkpoint inhibitor therapy, or other therapies known to a person of ordinary skill in the art.

If the adjunct therapy is of a modality suitable for administration by the same route as the fusion protein and essentially simultaneously with the fusion protein, the pharmaceutical composition can comprise the adjunct therapy.

In some embodiments, the adjunct therapy is a cell therapy or a gene therapy.

### Medical Uses of the Fusion Proteins

In some embodiments, the present disclosure relates to a composition or a fusion protein described herein for use in the treatment of a disease or disorder described herein.

In some embodiments, the present disclosure relates to the use of a composition or a fusion protein described herein in the treatment of a disease or disorder described herein.

In some embodiments, the present disclosure relates to the use of a composition or a fusion protein described herein in the manufacture of a medicament. The medicament can be for the treatment of a disease or disorder described herein.

### v. Methods for Stimulating Proliferation and/or Activation of Treg Cells

In some embodiments, the present disclosure relates to methods for stimulating proliferation and/or activation of T-regulatory (Treg) cells of a subject. The stimulation can be performed in vivo or in vitro.

For example, one in vitro method comprises isolating T cells from the subject; and contacting the isolated T cells with an effective amount of a composition or a fusion protein as described herein.

### vi. Kits

In some embodiments, the present disclosure relates to kits, comprising a composition or a fusion protein as described herein and instructions for performing a method as described herein.

For example, a kit can comprise a pharmaceutical composition as described herein; and instructions for performing a method for treating a disease or disorder characterized by inflammation, the method comprising administering to a subject in need thereof an effective amount of the pharmaceutical composition.

The instructions can be packaged in the kit in the form of printed instructions, a printed document providing a uniform resource locator (URL) from which detailed instructions can be browsed or downloaded, a printed document providing a Quick Response (QR) code which can be scanned to direct a smartphone or tablet computer's browser to a URL, or the like.

Kits will generally include one or more vessels or containers so that some or all of the individual components and reagents, e.g., the pharmaceutical composition, can be separately housed. Kits can also include a means for enclosing individual containers in relatively close confinement for commercial sale, e.g., a plastic box, in which instructions, packaging materials such as Styrofoam, etc., can be enclosed. An identifier, e.g., a bar code, radio frequency identification (ID) tag, etc., can be present in or on the kit or in or one or more of the vessels or containers included in the kit. An identifier can be used. e.g., to uniquely identify the kit for purposes of quality control, inventory control, tracking, movement between workstations, etc.

### B. SEQUENCES

| | | |
|---|---|---|
| SEQ ID NO: 1 | truncated wild | SITGISPITEYLASLSTYNDQSITFALEDESYEIYV |
| | type human IL- | EDLKKDEKKDKVLLSYYESQHPSNESGDGVDG |
| | 33 | KMLMVTLSPTKDFWLHANNKEHSVELHKCEK |
| | | PLPDQAFFVLHNMHSNCVSFECKTDPGVFIGVK |
| | | DNHLALIKVDSSENLCTENILFKLSET |
| SEQ ID NO: 2 | truncated N60S | SITGISPITEYLASLSTYNDQSITFALEDESYEIYV |
| | human IL-33 | EDLKKDEKKDKVLLSYYESQHPSSESGDGVDG |
| | | KMLMVTLSPTKDFWLHANNKEHSVELHKCEK |
| | | PLPDQAFFVLHNMHSNCVSFECKTDPGVFIGVK |
| | | DNHLALIKVDSSENLCTENILFKLSET |
| SEQ ID NO: 3 | truncated C116F | SITGISPITEYLASLSTYNDQSITFALEDESYEIYV |
| | human IL-33 | EDLKKDEKKDKVLLSYYESQHPSNESGDGVDG |
| | | KMLMVTLSPTKDFWLHANNKEHSVELHKCEK |
| | | PLPDQAFFVLHNMHSNFVSFECKTDPGVFIGVK |
| | | DNHLALIKVDSSENLCTENILFKLSET |
| SEQ ID NO: 4 | truncated C97G | SITGISPITEYLASLSTYNDQSITFALEDESYEIYV |
| | human IL-33 | EDLKKDEKKDKVLLSYYESQHPSNESGDGVDG |
| | | KMLMVTLSPTKDFWLHANNKEHSVELHKGEK |
| | | PLPDQAFFVLHNMHSNCVSFECKTDPGVFIGVK |
| | | DNHLALIKVDSSENLCTENILFKLSET |
| SEQ ID NO: 5 | truncated | SITGISPITEYLASLSTYNDQSITFALEDESYEIYV |
| | N60S + C116F | EDLKKDEKKDKVLLSYYESQHPSSESGDGVDG |
| | human IL-33 | KMLMVTLSPTKDFWLHANNKEHSVELHKCEK |
| | | PLPDQAFFVLHNMHSNFVSFECKTDPGVFIGVK |
| | | DNHLALIKVDSSENLCTENILFKLSET |
| SEQ ID NO: 6 | truncated | SITGISPITEYLASLSTYNDQSITFALEDESYEIYV |
| | N60S+C97G | EDLKKDEKKDKVLLSYYESQHPSSESGDGVDG |
| | human IL-33 | KMLMVTLSPTKDFWLHANNKEHSVELHKGEK |
| | | PLPDQAFFVLHNMHSNCVSFECKTDPGVFIGVK |
| | | DNHLALIKVDSSENLCTENILFKLSET |
| SEQ ID NO: 7 | truncated | SITGISPITEYLASLSTYNDQSITFALEDESYEIYV |
| | C116F+C97G | EDLKKDEKKDKVLLSYYESQHPSNESGDGVDG |
| | human IL-33 | KMLMVTLSPTKDFWLHANNKEHSVELHKGEK |
| | | PLPDQAFFVLHNMHSNFVSFECKTDPGVFIGVK |
| | | DNHLALIKVDSSENLCTENILFKLSET |
| SEQ ID NO: 8 | truncated | SITGISPITEYLASLSTYNDQSITFALEDESYEIYV |
| | N60S + C116F + | EDLKKDEKKDKVLLSYYESQHPSSESGDGVDG |
| | C97G human IL- | KMLMVTLSPTKDFWLHANNKEHSVELHKGEK |
| | 33 | PLPDQAFFVLHNMHSNFVSFECKTDPGVFIGVK |
| | | DNHLALIKVDSSENLCTENILFKLSET |
| SEQ ID NO: 9 | linker sequence: | GGGGS |
| SEQ ID NO: 10 | linker sequence | GGGGSGGGGSGGGGS |
| SEQ ID NO: 11 | signal peptide | MGWSCIILFLVATATGVHS |
| SEQ ID NO: 12 | human IL-2 with | MYRMQLLSCIALSLALVTNSAPTSSSTKKTQLQ |
| | signal peptide | LEHLLLDLQMILNGINNYKNPKLTRMLTFKFY |
| | | MPKKATELKHLQCLEEELKPLEEVLNLAQSKN |
| | | FHLRPRDLISNINVIVLELKGSETTFMCEYADET |
| | | ATIVEFLNRWITFCQSIISTLT |
| SEQ ID NO: 13 | wild type human | APTSSSTKKTQLQLEHLLLDLQMILNGINNYKN |
| | IL-2 | PKLTRMLTFKFYMPKKATELKHLQCLEEELKP |
| | | LEEVLNLAQSKNFHLRPRDLISNINVIVLELKGS |
| | | ETTFMCEYADETATIVEFLNRWITFCQSIISTLT |
| SEQ ID NO: 14 | human IL-2 | APTSSSTKKTQLQLEHLLLDLQMILNGINNYKN |
| | N88D | PKLTRMLTFKFYMPKKATELKHLQCLEEELKP |
| | | LEEVLNLAQSKNFHLRPRDLISDINVIVLELKGS |
| | | ETTFMCEYADETATIVEFLNRWITFCQSIISTLT |
| SEQ ID NO: 15 | human IL-2 | APTSSSTKKTQLQLEHLLLDLQMILNGINNYKN |
| | C125S | PKLTRMLTFKFYMPKKATELKHLQCLEEELKP |
| | | LEEVLNLAQSKNFHLRPRDLISNINVIVLELKGS |
| | | ETTFMCEYADETATIVEFLNR WITFSQSIISTLT |
| SEQ ID NO: 16 | human IL-2 | APTSSSTKKTQLQLEHLLLDLQMILNGINNYKN |
| | N88D + C125S | PKLTRMLTFKFYMPKKATELKHLQCLEEELKP |
| | | LEEVLNLAQSKNFHLRPRDLISDINVIVLELKGS |
| | | ETTFMCEYADETATIVEFLNRWITFSQSIISTLT |

### 7. EQUIVALENTS

It will be readily apparent to those skilled in the art that other suitable modifications and adaptions of the methods of the invention described herein are obvious and can be made using suitable equivalents without departing from the scope of the disclosure or the embodiments. Having now described certain compositions and methods in detail, the same will be more clearly understood by reference to the following examples, which are introduced for illustration only and no intended to be limiting.

### 8. EXAMPLES

The following are examples of methods and compositions of the invention. It is understood that various other embodiments can be practiced, given the general description provided herein.

TABLE 1

| | |
|---|---|
| IL-233 | Fusion cytokine of IL-2-linker-IL33 made from rodent |
| | WT versions (mouse or rat as appropriate) |
| SLT518 | Generic name for fusion cytokine of IL-2-linker-IL-33 |
| SLT518(r) | Rat version for fusion cytokine of IL-2-linker- IL-33 |
| SLT518(m) | Mouse version of fusion cytokine of IL-2-linker-IL33 |
| SLT518(h#) | Human version and specific variant of fusion |
| | cytokine of IL-2-linker-IL-33 |
| SLT518(h4) | Human version of IL-2 mutein/IL-33 fusion |
| | tool molecule |
| ST2 | Receptor for IL-33 |
| IL-33R | Receptor for IL-33 (more commonly referred to as ST2) |

### Example 1: Effect of N60D Mutation on Activity

The effect of an N60D point substitution in a truncated IL-33 domain on the IL-2 activity of a fusion protein comprising an IL-2 domain and a truncated IL-33 domain was investigated. Specifically, the fusion protein was synthesized as a single polypeptide by recombinant cells, purified, and a sample was assessed for IL-2 activity using a known assay, with activity measured by OD_{620 nm}. IL-2 alone was used as a positive control. IL-33 alone was used as a negative control.

FIG. 3 shows the results. The fusion protein containing an N60D substitution in the truncated IL-33 domain retained essentially the same IL-2 activity as IL-2 alone.

### Example 2: Effect of C97G Mutation on Activity

The effect of a C97G point substitution in a truncated IL-33 domain on the IL-2 activity and the IL-33 activity of a fusion protein comprising an IL-2 domain and a truncated IL-33 domain was investigated. Specifically, the fusion protein was synthesized as a single polypeptide by recombinant cells, purified, and a sample was assessed for IL-2 activity or IL-33 activity using known assays, with activity measured by OD_{620 nm}. In the IL-2 activity assay, IL-2 alone was used as a positive control and IL-33 alone was used as a negative control. In the IL-33 activity assay, IL-33 alone was used as a positive control and IL-2 alone was used as a negative control.

FIG. 4 shows the results of the IL-2 activity assay. The fusion protein containing a C97G point substitution in the truncated IL-33 domain retained essentially the same IL-2 activity as IL-2 alone.

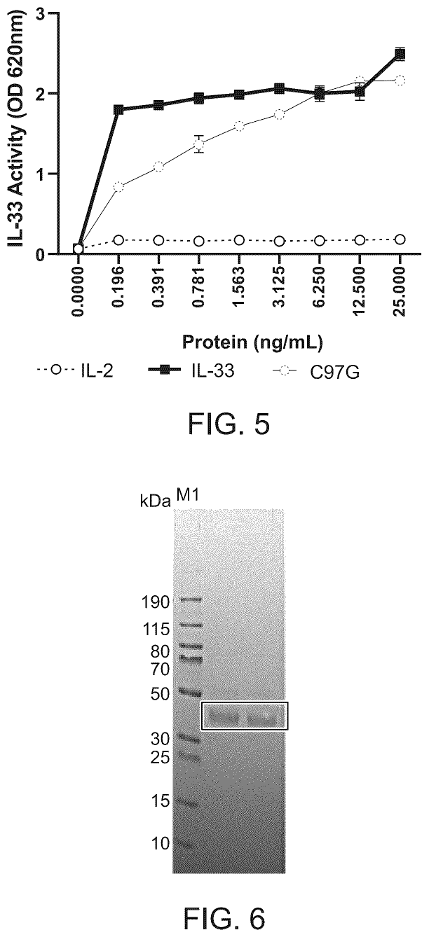

FIG. 5 shows the results of the IL-33 activity assay. The fusion protein containing a C97G point substitution in the truncated IL-33 domain showed comparable IL-33 activity as to IL-33 alone.

### Example 3: Effect of C116F Mutation on Expression, Activity and Stability

The effect of a C116F point substitution in a truncated IL-33 domain on the expression, purification, IL-2 activity, IL-33 activity, and stability of a fusion protein comprising both an IL-2 domain and a truncated IL-33 domain was investigated. Specifically, the fusion protein was synthesized as a single polypeptide by recombinant cells, purified, and samples were assessed for IL-2 activity and IL-33 activity after 0 days, 3 days, or 7 days of storage at 4° C., 25° C., or 37° C. IL-2 and IL-33 activity were measured as described above.

FIG. 6 shows the fusion protein containing a C116F substitution in the truncated IL-33 domain was well expressed in the recombinant cells. The thick bands in the center and right columns correspond to the molecular weight of the fusion protein.

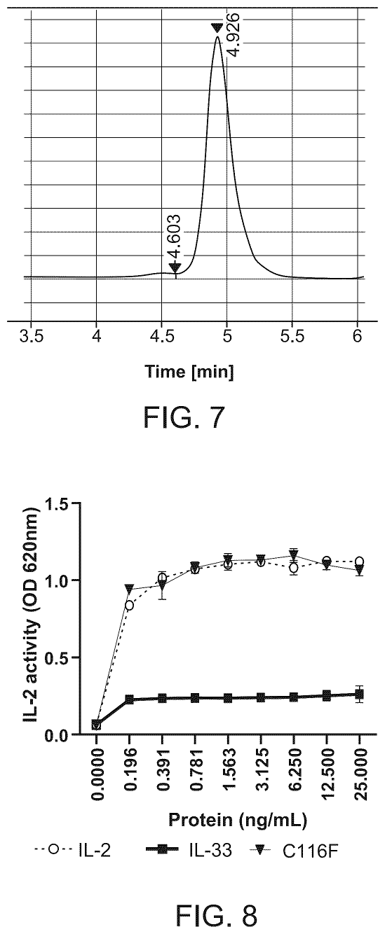

FIG. 7 shows the fusion protein containing a C116F substitution in the truncated IL-33 domain was easily purified by standard chromatography techniques.

FIG. 8 shows the fusion protein containing a C116F substitution in the truncated IL-33 domain retained essentially the same IL-2 activity as IL-2 alone.

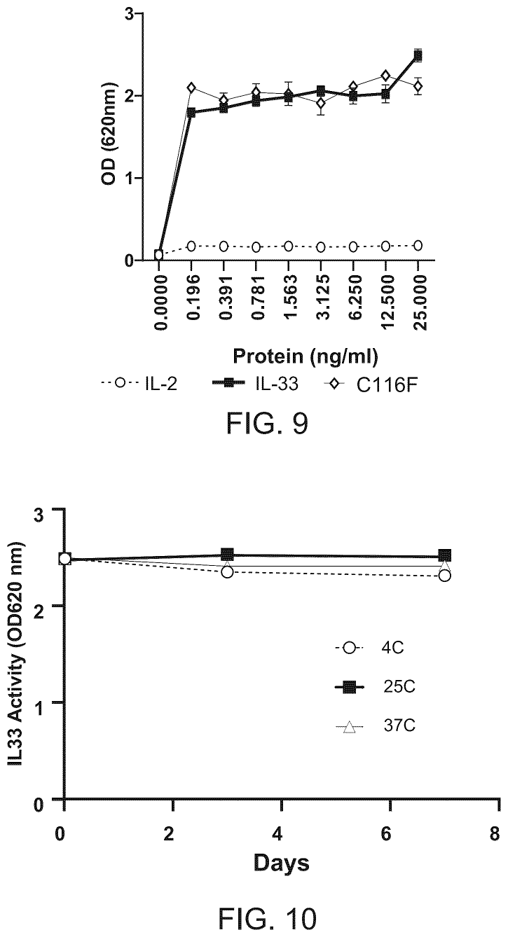

FIG. 9 shows the fusion protein containing a C116F substitution in the truncated IL-33 domain retained essentially the same IL-33 activity as IL-33 alone.

FIG. 10 shows the fusion protein containing a C116F substitution in the truncated IL-33 domain retained at least about 90% or more of its initial IL-33 activity, even after 7 days of storage at 37° C.

### Example 4: Activity of Multiple Engineered Constructs

TABLE 2

| SLT- | IL 2 | IL2 | IL33 | IL33 | IL33 |
|---|---|---|---|---|---|
| 518 | N88D | C125S | N60S | C116F | C97G |
| h1 (SLT1) | | | | | |
| h2 (SLT2) | X | | | | |
| h3 (SLT3) | X | | X | | |
| h4 (SLT4) | X | | | X | |
| h5 (SLT5) | X | | | | X |
| h6 (SLT6) | X | | X | X | X |
| h7 (SLT7) | X | | | X | X |

| | | | | |
|---|---|---|---|---|
| h8* (SLT8) | X | X | X | X |
| h9 (SLT9) | X | X | X | |

Nine engineered constructs were made with various mutations in IL2 and/or IL33 (SLT-518(h1), SLT-518(h2), SLT-518(h3), SLT-518(h4), SLT-518(h5), SLT-518(h6), SLT-518(h7), SLT-518(h8), and SLT-518(h9). The details of the mutations contained within each construct are provided in the above Table 2. The rationally designed variants were produced to optimize expression, activity, and stability, while retaining wild type sequences to limit immunogenicity risk.

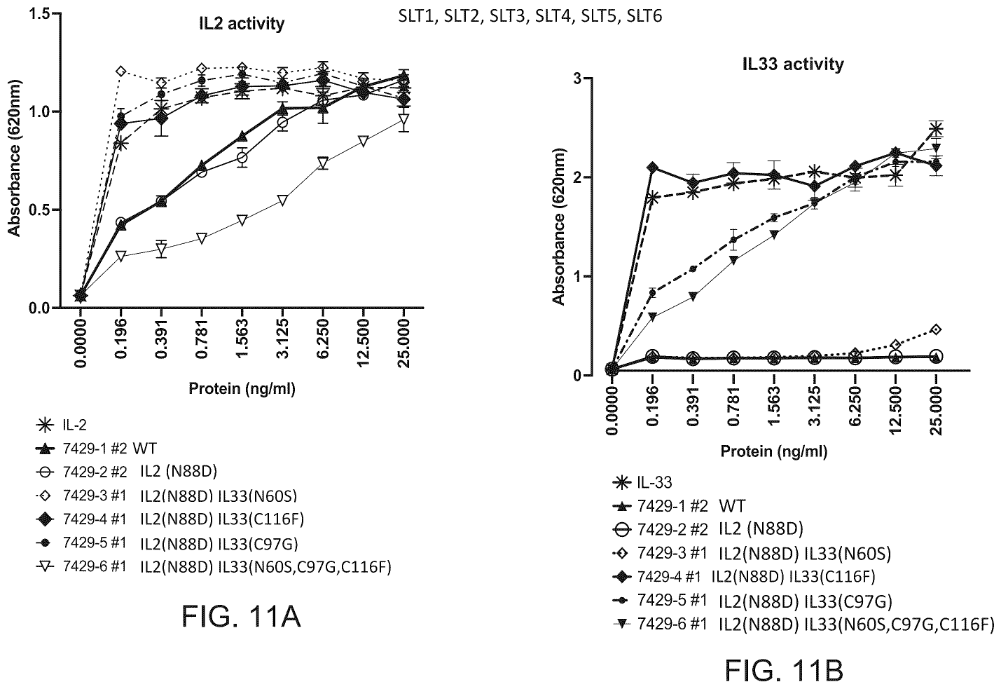

IL2 and IL33 activity was measured for six of the engineered variants (SLT1,SLT2, SLT3, SLT4, SLT5, SLT6) in FIGS. 3-5. In FIGS. 11-14 the SLT-518 constructs are referred to as 7429-(variant #), corresponding to (SLT-518(h1), SLT-518(h2), SLT-518(h3), SLT-518(h4), SLT-518(h5), or SLT-518(h6). SLT1 and SLT2 have minimal IL33 activity and compared to SLT3, SLT4, SLT5 and SLT6.

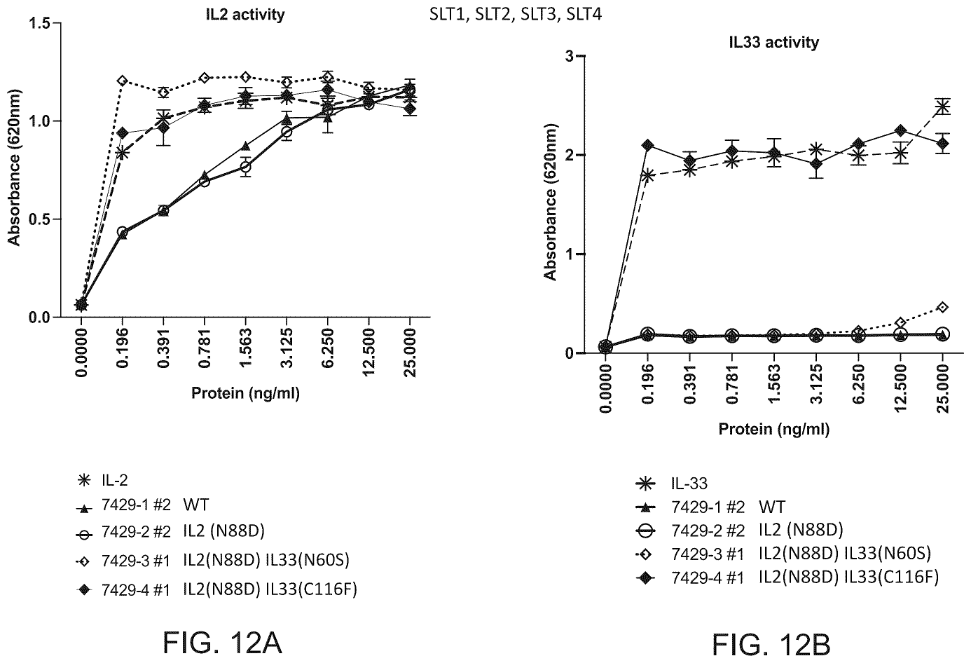

SLT3 (an IL233 fusion protein containing IL2(N88D) and IL33(N60S) mutations) and SLT4 (an IL233 fusion protein containing IL2(N88D) and IL33(C116F) mutations) was compared to SLT1 (wild type fusion protein IL233) and SLT2 (an IL233fusion protein containing an IL2(N88D) mutation (FIG. 12A and FIG. 12B). As seen in FIG. 11A and FIG. 11B, SLT1 and SLT2 IL33 activity is barely detectable. SLT3 and SLT4 have significantly greater IL33 activity, as compared SLT1 and SLT2.

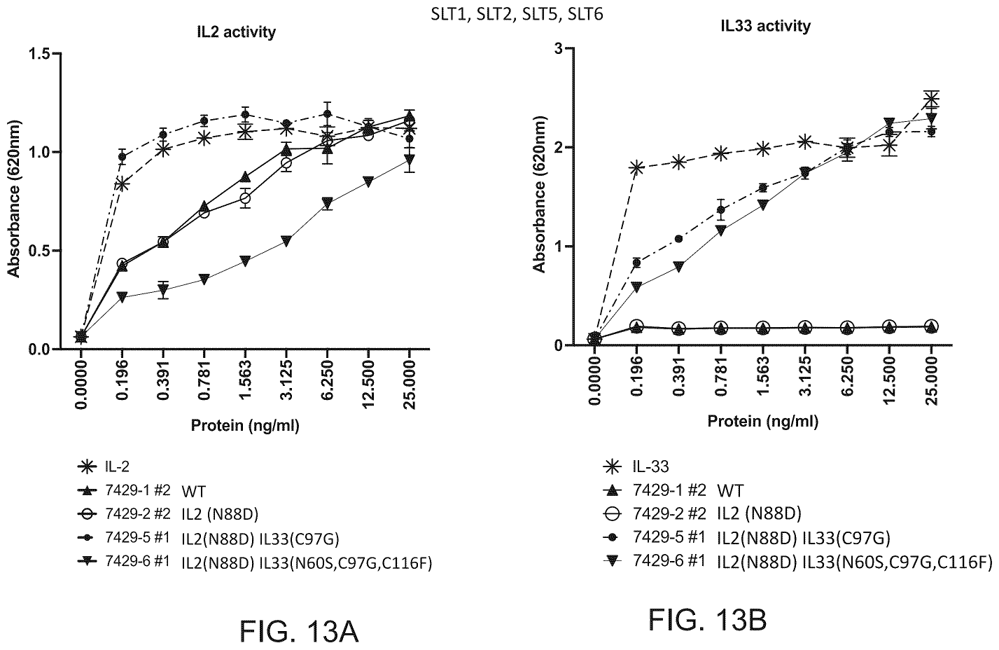

SLT5 (an IL233 fusion protein containing IL2(N88D) and IL33(C97G) mutations) and SLT6 (an IL233 fusion protein containing IL2(N88D) and IL33(N60S, C97G, C116F) mutations) was compared to SLT1 (wild type fusion protein IL233) and SLT2 (an IL233 fusion protein containing an IL2(N88D) mutation (FIG. 13A and FIG. 13B). As seen above, SLT1 and LST2 IL33 activity is minimal. SLT5 and SLT6 have significantly greater IL33 activity, as compared to SLT1 and SLT2.

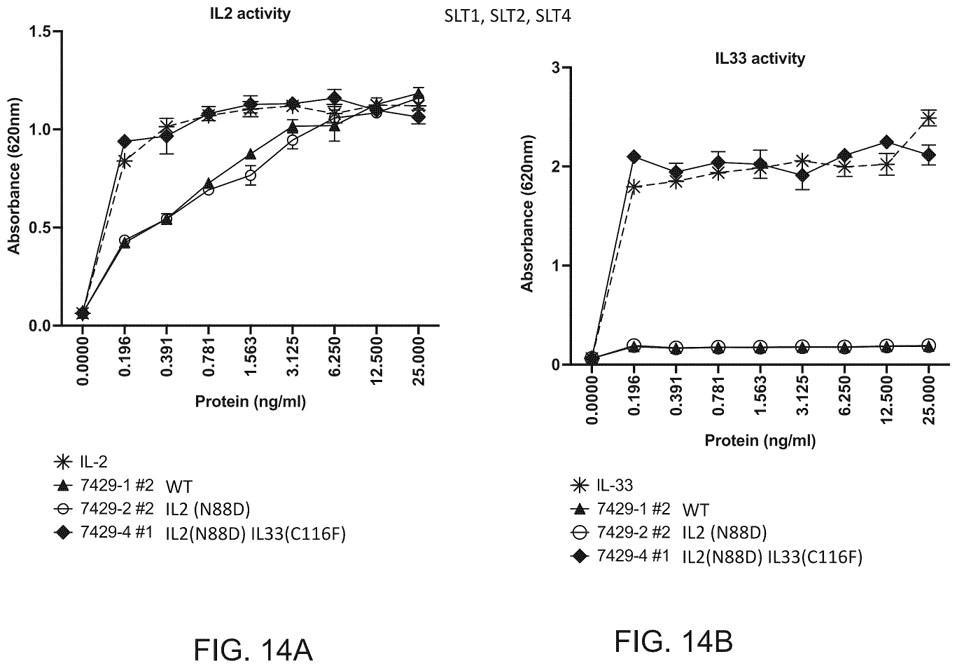

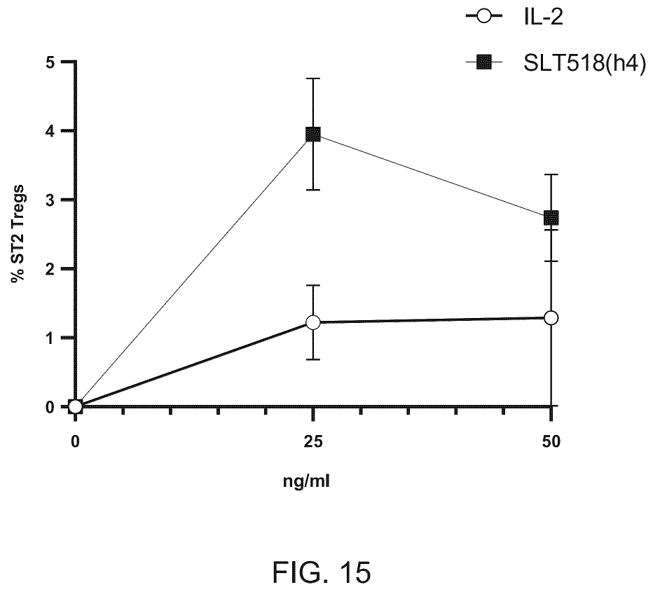

SLT4 (an IL233 fusion protein containing IL2(N88D) and IL33(C116F) mutations) was compared to SLT1 and SLT2 (FIG. 14A and FIG. 14B). SLT4 achieves approximately the same level of IL2 activity as compared to wildtype IL2, while also achieving approximately the same IL33 activity level as the wildtype IL33.

### Example 5: Tissue Targeting and ST2+ Treg Cells

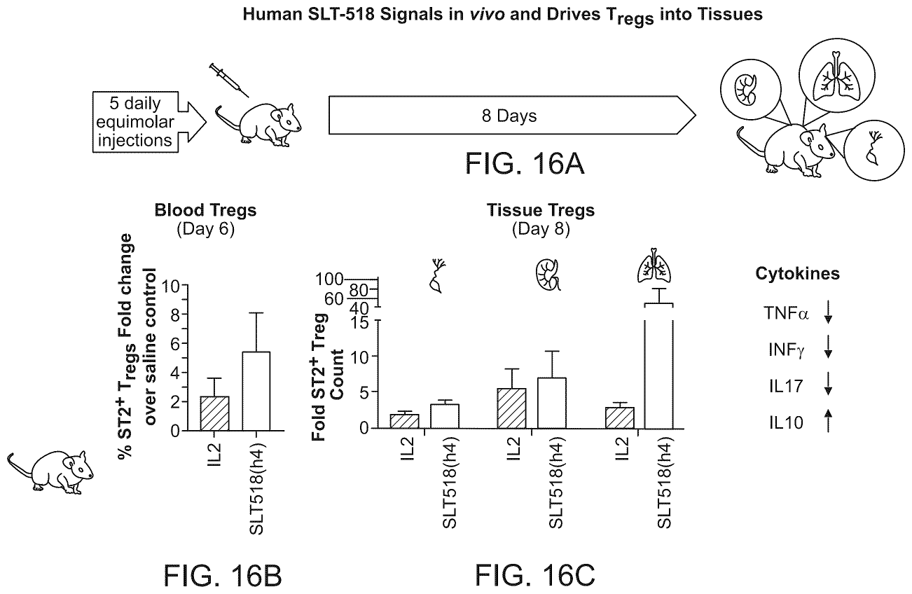

Healthy mice were treated with 66 pmol of IL233 fusion protein (SLT-518(h4)) daily for five days. Tissues were harvested on Day 8. The average ST2 expression increased in Tregs. The total number of ST2+ Tregs increased in the spleen. The proportion of ST2+ cells increased in the Treg population. Thus, in the tissue microenvironment, SLT-518(h4) upregulates the highly suppressive ST2+ Tregs (FIG. 16A, FIG. 16B and FIG. 16C).

SLT-518(h4) stimulated active mobilization of Tregs to injured tissue via IL-33 dependent signals in the tissue microenvironment in an ischemic kidney injury model.

In summary, SLT518(h4) was shown to drive the expression of ST2+ Treg cells in a healthy mouse. SLT518(h4) can accelerate mobilization of the ST2+ Treg cell to sites of injury, which means that systemic administration of this therapeutic can drive a local effect. Tregs from SLT518(h4) are more potent on a per cell basis to suppress T-cell proliferation in vitro and the disease in vivo.

The data presented herein with SLT518(h4) shows that the newly discovered mutations improve the function and stability of the molecule, as compared to the previously published mouse protein.

### Example 6: Tissue Microenvironment and Durability

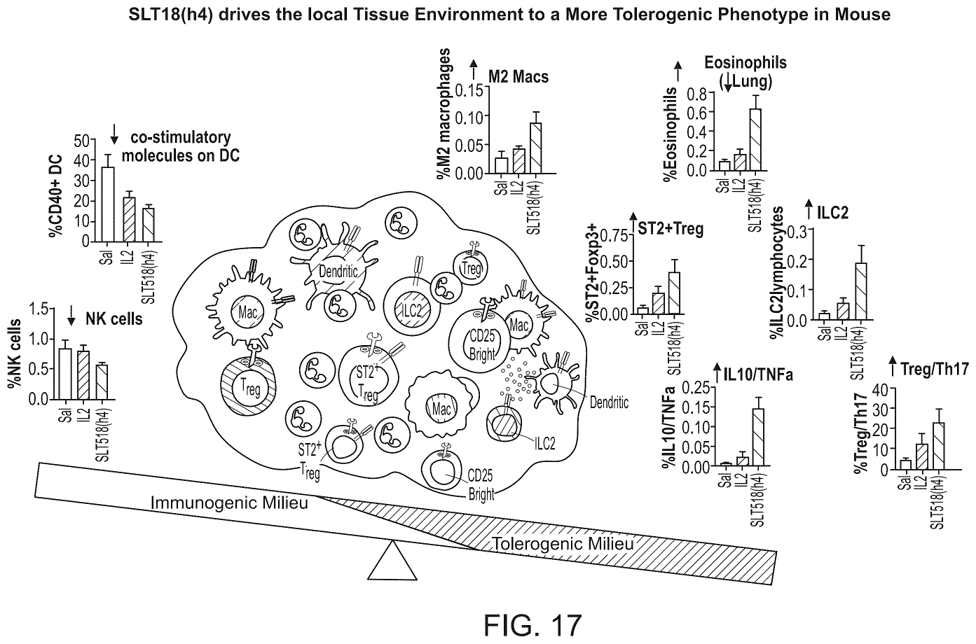

Durable immune responses require more than just Tregs. Overlooking the other cells in the tissue micro-environment can lead to temporary, less potent effects and does not address the underlying etiology of disease. Prior studies have shown that a low dose of IL-2 targets primarily Tregs, which produces only a temporary response. A low dose of IL-2+IL33, however, targets multiple cell types, including IL-2 producing tolerogenic dendritic cells (tolDCs) (FIG. 17). Thus, an IL233 fusion cytokine restores the tissue microenvironment to establish long term tolerogenic and reparative states. The effect of a restored microenvironment is a durable effect, without the need for additional therapeutic input.

### Example 7: Cynomolgus Monkey Studies

Healthy non-human primate studies were conducted at Wuxi Apptec (Shanghai, PRC) using SLT-518(h4) derived from CHO manufacturing process developed at Wuxi Biologics (Shanghai, PRC). All comparisons of SLT-518(h4) and IL-2 were equimolar dosing five daily doses to match Proleukin as a benchmark.

The comprehensive cynomolgus monkey studies evaluated over 35 individual animals with escalating doses. Systemic pharmacodynamics (as Treg and Treg subsets), local pharmacodynamics (as Treg and Treg subsets in lymph node, tissue TBD), tolerability, safety and selectivity index (as Treg/Teff subsets), pharmacokinetics (single and repeat dose) were evaluated. Different routes of administration were tested, as were multiple comparator products.

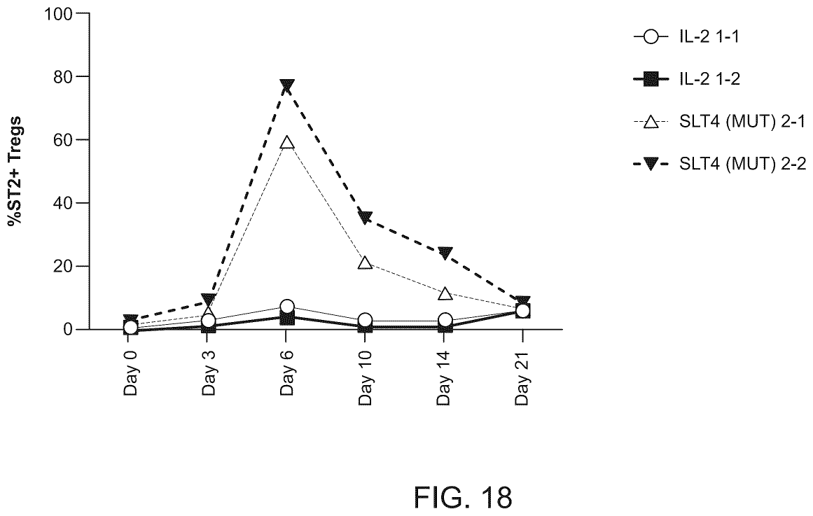

Treatment with SLT-518(h4) promoted dramatic expansion of ST2+ Treg cells in healthy non-human primates (FIG. 18). Two monkeys were dosed with SLT518(h4) and two monkeys were dosed with IL-2 alone for 21 days. The percentage of ST2+ Tregs were measured, and more than 3000-fold ST2+ Tregs were measured at day 6 in the monkeys dosed with an engineered IL233 fusion protein (SLT-518(h4)).

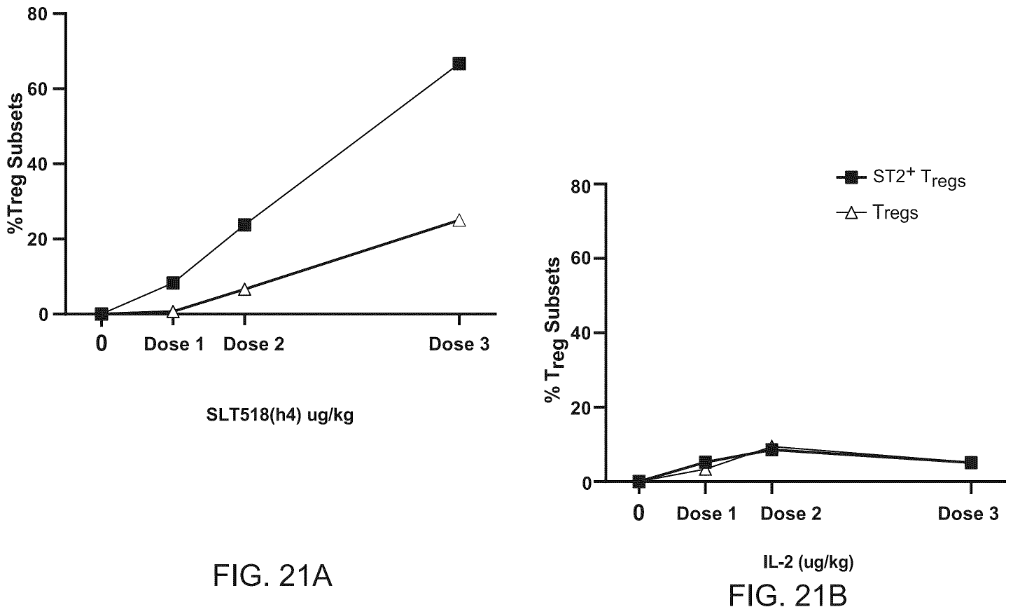

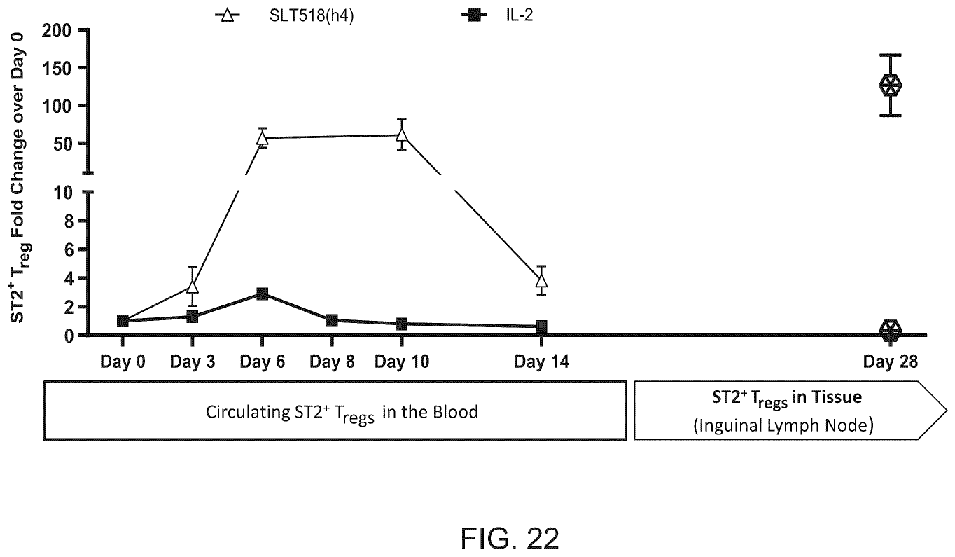

SLT-518(h4) also induced superior expansion of ST2+ Tregs, thereby providing a wider therapeutic margin relative to clinical doses of IL-2 (FIGS. 21A and 21B). Three doses of SLT-518(h4) were administered to the monkeys (FIG. 21A), and three doses of IL-2 alone were administered to the monkeys (FIG. 21B). The percentage of Treg subsets were measured and tracked against a target clinical range.

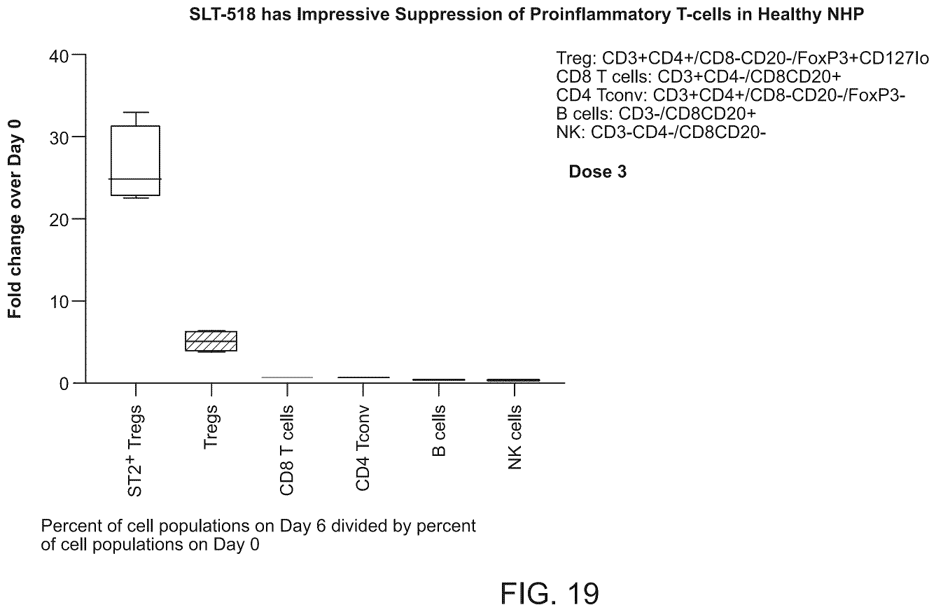

SLT-518(h4) also exhibited a dramatic expansion of ST2+ Tregs in the blood and remained elevated in the tissue for over 28 days, well beyond the levels observed in the blood (FIG. 19).

In summary, mutation of C116F resulted in a humanized form of IL233 (SLT518(h4)) with superior activity over wildtype versions, as shown by the comparative data wherein SLT518(h1) and SLT518(h2) were compared to SLT518(h4) in both in vitro and non-human primate studies.

### Example 8: Non-Human Primate Tregs

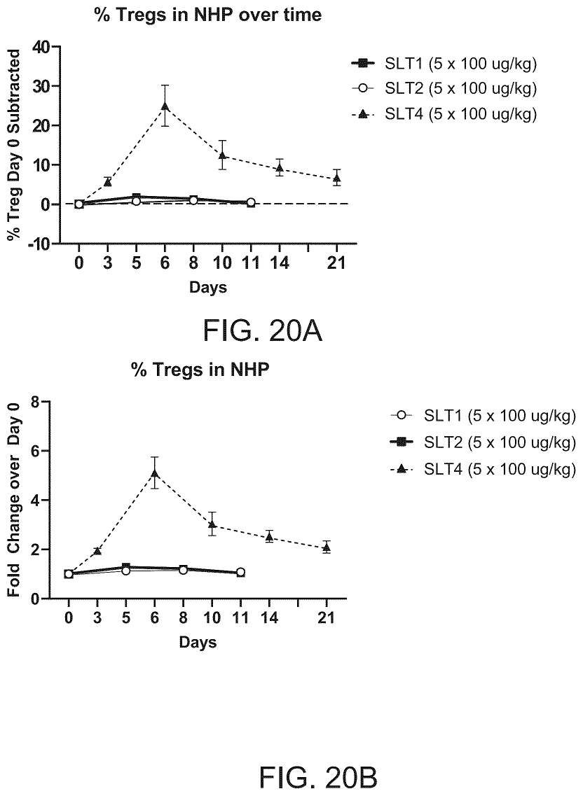

Non-human primates were administered a 100 ug/kg dose of either SLT1(wild type fusion protein IL233), SLT2 (an IL233 fusion protein containing an IL2(N88D) mutation) or SLT4 (an IL233 fusion protein containing IL2(N88D) and IL33(C116F) mutations) daily for five days. The percentage of Tregs were measured for 21 days. FIG. 20A and FIG. 20B shows that SLT4 produced a dramatic increase in the percentage of Tregs and the fold change over day 0 in the non-human primates, peaking at day 6.

Similar to the mouse data shown above, SLT518(h4) (indicated as SLT4 in FIG. 20A and FIG. 20B) was shown to drive the expression of ST2+ Treg cells in non-human primates and performed significantly better than SLT518(h1) (indicated as SLT1) and SLT518(h2) (indicated as SLT2). This data indicates that the key mutation for increased expression of ST2+ Treg cells is owed to the IL33 C116F mutation.

What is claimed:

1. A composition, comprising:
an interleukin-2 (IL-2) having at least 90% identity to SEQ ID NO: 13; and
a truncated interleukin-33 (IL-33), comprising a sequence with at least 90% identity to SEQ ID NO: 1.

2. A composition, comprising:
an interleukin-2 (IL-2) having the amino acid sequence of SEQ ID NO: 14; and a truncated interleukin-33 (IL-33), having the amino acid sequence of SEQ ID NO: 3.

3. A fusion protein, comprising:
an interleukin-2 (IL-2) domain having at least 90% identity to SEQ ID NO: 13; and
a truncated interleukin-33 (IL-33) domain, comprising a sequence with at least 90% identity to SEQ ID NO: 1.

4. A fusion protein, comprising:
an interleukin-2 (IL-2) having the amino acid sequence of SEQ ID NO: 14; and
a truncated interleukin-33 (IL-33), having the amino acid sequence of SEQ ID NO: 3.

5. The composition of claim 1 or the fusion protein of claim 3, wherein in the truncated IL-33 or the truncated IL-33 domain, at least one of N60, C97, C116, C121, and C148 of SEQ ID NO: 1 is substituted by a single amino acid.

6. The composition of claim 1 or the fusion protein of claim 3 or 5, wherein N60 of SEQ ID NO: 1 is substituted by Ser or Asp, C97 of SEQ ID NO: 1 is substituted by Gly, or C116 of SEQ ID NO: 1 is substituted by Phe.

7. The composition of claim 1 or the fusion protein of any one of claims 3 to 6, wherein the truncated IL-33 or the truncated IL-33 domain has a sequence selected from the group consisting of SEQ ID NO: 3, SEQ ID NO: 4, and SEQ ID NO: 7.

8. The fusion protein of any one of claims 3 to 7, further comprising a linker.

9. The fusion protein of claim 8, wherein the linker comprises GGGGS (SEQ ID NO: 9).

10. The fusion protein of claim 8 or 9, wherein the linker has the sequence
(SEQ ID NO: 10)
GGGGSGGGGSGGGGS.

11. The composition of claim 1 or the fusion protein of any one of claims 3 to 10, wherein the IL-2 or the IL-2 domain has a sequence selected from the group consisting of SEQ ID NO: 13, SEQ ID NO: 14; SEQ ID NO: 15; and SEQ ID NO: 16.

12. The fusion protein of any one of claims 3 to 11, further comprising a signal peptide.

13. The fusion protein of claim 12, wherein the signal peptide has the sequence of SEQ ID NO: 11.

14. The fusion protein of any one of claims 3 to 13, wherein the domains are, in order from N-terminus to C-terminus, an optional signal peptide; the interleukin-2 (IL-2) domain; an optional linker; and the truncated interleukin-33 (IL-33) domain.

15. A pharmaceutical composition, comprising the composition of claim 1 or 2 or the fusion protein of any one of claims 3 to 14.

16. The pharmaceutical composition of claim 15, further comprising a pharmaceutically-acceptable carrier, and optionally an additional therapeutic agent.

17. A polynucleotide comprising a nucleic acid sequence encoding at least a portion of the fusion protein of any one of claims 3 to 14.

18. A vector comprising the polynucleotide of claim 17 operatively coupled to a promoter.

19. A recombinant host cell comprising the polynucleotide of claim 17 or the vector of claim 18.

20. A method for treating a disease or disorder, comprising:
administering to a subject in need thereof an effective amount of the pharmaceutical composition of claim 15 or 16.

21. The method of claim 20, wherein the disease or disorder is selected from the group consisting of acute kidney injury, ankylosing spondylitis, autoimmune cardiomyopathy, autoimmune hemolytic anemia, autoimmune hepatitis, autoimmune lymphoproliferative syndrome, autoimmune pancreatitis, autoimmune polyendocrine syndrome, autoimmune urticaria, autoimmune uveitis, Crohn's disease, dermatomyositis, diabetic nephropathy, diabetic retinopathy, graft versus host (GVH) disease, Hashimoto's thyroiditis, idiopathic inflammatory bowel disease (IBD), inflammatory demyelinating diseases, Inflammatory neuropathies, insulitis, interstitial cystitis, juvenile idiopathic arthritis, lupus, lupus erythematosus, lupus glomerulonephritis, IgA nephropathy, membranous nephropathy (MPGN), microscopic colitis, multiple sclerosis, myasthenia gravis, obesity, pancreatitis, polymyositis, primary biliary cirrhosis, primary sclerosing cholangitis, progressive inflammatory neuropathy, renal ischemia reperfusion injury, rheumatoid arthritis, Sjogren's syndrome, systemic lupus erythematosus, transplant rejection, type 1 diabetes, type 2 diabetes, ulcerative colitis, vasculitis, and Wegener's granulomatosis.

22. The method of claim 20, wherein the disease or disorder is a cancer.

23. The method of claim 22, wherein the cancer is selected from the group consisting of acute lymphoblastic leukemia (ALL), acute myeloid leukemia (AML), acute myelogenous leukemia, adrenocortical carcinoma, anal cancer, appendix cancer, astrocytoma, basal cell carcinoma, B-cell lymphoma, brain cancer, bile duct cancer, bladder cancer, bone cancer, breast cancer, bronchial tumor, carcinoma of unknown primary origin, cardiac tumor, cervical cancer, chordoma, chronic lymphocytic leukemia, chronic myelogenous leukemia, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphocytic leukemia, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

24. The method of claim 23, wherein the cancer is selected from the group consisting of melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, and lung cancer.

25. The method of any one of claims 20 to 24, wherein the method stimulates proliferation and/or activation of T-regulatory (Treg) cells, T-helper2 (Th2) cells, innate lymphoid cells (ILC), cytotoxic T cells, natural killer (NK) cells, NK-T cells, ST2+ cells, dendritic cells, and/or macrophages.

26. The method of any one of claims 20 to 25, wherein the method promotes anti-inflammatory M2 macrophages and inhibits pro-inflammatory M1 macrophages.

27. The method of claim 26, wherein the M2 macrophages inhibit inflammation in a tissue or organ selected from the group consisting of pancreas, liver, kidneys, adipose tissue, salivary glands, central nervous system (CNS), and related organs.

28. The method of any of claims 20-24, wherein the method, depending on the IL-2 domain, the method can either upregulate or down-regulate the expression of co-stimulatory molecules (including and not limited to CD80 (B7-1), CD86 (B7-2), CD40, ICOS, MHC-I, MHC-II, PD1, PD-L1, GITR, BAFF-R, Ox40, 41BB, DR3, CR2) on the antigen-presenting cells including and not limited to dendritic cells, macrophages, B-cells, innate lymphoid cells, NK cells, epithelial cells, endothelial cells or stromal cells.

29. The method of any one of claims 20 to 27, further comprising:
administering an effective amount of a second therapy for the disease or disorder to the subject, wherein the second therapy does not comprise the composition of claim 1 or 2 or the fusion protein of any one of claims 3 to 14.

30. The method of claim 29, wherein the second therapy is a cell therapy or a gene therapy.

31. A method for stimulating proliferation and/or activation of T-regulatory (Treg) cells of a subject, comprising:
isolating T cells from the subject; and
contacting the isolated T cells with an effective amount of the composition of claim 1; the fusion protein of any one of claims 3 to 14; a cell expressing at least one of IL-2 or truncated IL-33; or the recombinant host cell of claim 19.

32. A kit, comprising:
the pharmaceutical composition of claim 15 or 16; and
instructions for performing a method for treating a disease or disorder characterized by inflammation, the method comprising administering to a subject in need thereof an effective amount of the pharmaceutical composition.
